(19) United States
(12) Patent Application Publication
(10) Pub. No.: US 2024/0371530 A1
(43) Pub. Date: Nov. 7, 2024

(54) Adaptive Selection of Messages for Transmission in Networked Environments to Increase Session Adherence

(71) Applicant: Click Therapeutics, Inc., New York, NY (US)

(72) Inventors: Sudheer Guttikonda, Edison, NJ (US); Gunther Havel, Portsmouth, NH (US); Patrick Tan, Jersey City, NJ (US); William Morse, New York, NY (US)

(73) Assignee: Click Therapeutics, Inc., New York, NY (US)

(21) Appl. No.: 18/143,989

(22) Filed: May 5, 2023

(51) Int. Cl. G16H 80/00; G16H 20/70; G16H 40/20; H04L 67/50

(52) U.S. Cl. CPC G16H 80/00; G16H 20/70; G16H 40/20; H04L 67/535

(57) ABSTRACT

Provided herein are systems and methods of controlling the transmission of messages in a networked environment. A server can send a set of instructions to a user device associated with a user, each of the instructions to present a message in at least one of a set of variants. The server can receive a set of responses corresponding to the instructions, each of the responses by the user to a respective presentation of the message in at least one of the variants. The server can determine a respective performance measure of the message in each variant using the responses and can update a selection model responsive to the responses. The server can select variant from the based on the performance measure for the variant. The server can transmit a second instruction to the user device associated with the user to present the message in the selected variant.

### BACKGROUND

Poor adherence to medical treatments is a serious problem in the health care industry. Patients may find it difficult to adhere to treatments, leading to ineffective clinical outcomes and higher costs. Despite industry efforts, medication adherence rates have not changed significantly over time and have remained low. There are no effective and affordable methods of helping patients adhere to prescribed treatments. There is a need for a method to improve adherence among patients and ultimately influence clinical outcomes.

### SUMMARY

In a messaging service under the present disclosure, a service may select which variation of a message, among multiple variations, to send to a user. The variations may correspond to different types of content, theme, tone, or layout for a given message, or based on factors such as the user's personality, literacy, health knowledge, learning style, personal incentive or motivation, messaging style, or preference for inclusion of the user's name. The objective of selection may be to provide the variant that maximizes user engagement as measured by interaction rates. For message platforms in the digital therapeutics context, an additional objective may include adherence to a session to address a user's condition (e.g., behavior, psychological, or mental) as measured by various factors over a course of time. This way, the amount of computing resources and network bandwidth consumed in transmitting otherwise ineffective message variants may be avoided.

A challenge with other approaches in selecting variants of messages may be the initial lack of data (e.g., history or response data) about a given user. This may be because before the user begins engaging with the platform, the service may not have any information on the content preferences or typical behavior about the user with respect to interactions with messages. As a result, the service may have poor determinants for selecting which particular messages to transmit to the user device. Without any information, the service may end up selecting and sending message variants resulting in little to no user interaction, indicating low quality of human-computer interactions (HCI). This also may lead to wasted consumption of computing resources on both the service and user device as well as expenditure of network bandwidth.

In the context of digital therapeutics applications in other approaches, messages may be provided via the application on the user device to administer the treatment to the user, with the user in some cases being prompted to open the application to self-administer the therapeutic intervention. The service may have at most information on a condition of the user to be addressed, and little to no data on user performance in response to messages or adherence to the session. This lack of information on the part of the service may lead to the presentation with message variants with little to no relevance to the user. As a consequence, the user may experience message fatigue from repeated presentations of the little to no relevance to the user, resulting in lower adherence to the treatment regimen for the session. The low adherence to the session may in turn lead to no changes in or even worsening the condition of the user to be addressed through the digital therapeutics application.

One attempt to addressing these problems may be to implement A/B testing. For example, a message platform service may define groups of users, with one group of users to receive a message variant “A” and another group of users to receive message variant “B”. In this setup, the message platform service may then gather response data and measure the performance of each message variant from these groups of users during the testing phase. Afterwards, the service may select one of the variants to provide across all users based on the performance.

The A/B testing approach, however, may have a number of issues. For one, by selecting message variants for the entire set of users, it may be difficult to individualize selection of message variants for particular users. This may be so, even if certain message variants may have high rates of interaction with different users. For another, since individualization is not considered, the service may over-collect measurement of user responses over each group, leading to wasted consumption of computer resources and network bandwidth and poor quality of HCI. Furthermore, the A/B testing approach may be inappropriate for digital therapeutics applications, because users are limited to seeing the message variant for their own group. This may limit the customizability of sessions to address their conditions and leave the issue with low adherence to treatment unresolved.

To address these and other technical challenges, the service in the present disclosure may use a model for a particular user's responses to message variants to perform individualized selection for the given user. As the user begins engaging with the application, the service may establish a machine learning model (or a statistical model). The machine learning model may be, for example, a multi-armed bandit model updated according to Thompson sampling. The model may be used to define a selection probability for each message variant that is updated based on a performance metric indicated from the user's response to the message variant. As the service begins to gather data on the user behavior, certain user preferences may begin to emerge. The data may include, for example, whether the user interacted with the message variant, whether the user responded correctly to the message, and performance data with respect to the session to address the user's condition, among others.

In the context of digital therapeutic applications, the message variants (e.g., notifications) can have a causal effect on user behavior. For example, a message received from the service to the user device can cause a user to perform a task in a treatment regimen provided by the service. To facilitate ease and consistency for a user participating in a treatment regimen, the messaging application or platform running on the device may be used for generating and pushing messages to encourage the user to participate in sessions of the regimen. Sessions may be incorporated into an application on the user device to address a user's mental and physical goals. For example, an application executing on a user device can receive a message encouraging the user to participate in a task of their training regimen, and the user can subsequently participate in the task.

A message can include a mechanism for responding, such as a link (e.g., a uniform resource locator (URL)) within the message, a text box, or using a different platform or application also related to the application. For example, given an initial presentation of two types of variants of messages to a user, a user may indicate a preference for the first message over the second message by responding to the first message and not the second message. Based on the result of the message (e.g., if it elicited a response from the user), the prior probabilities can be updated. The service can then again randomly select a next message to send with the probability weighted by the updated priors. For example, the service can perform a Thompson sampling algorithm on a multitude of variants and corresponding responses to establish a set of prior probabilities for each variant of a message for a user. This iterative process can continue until the service is able to converge on user preferences or treatment ends.

Users may prefer certain messages relative to others. Messages transmitted to a user can be in different variants. For example, one or more different types or variants of a message can be transmitted to a user device corresponding to a task of the regimen. Users are more likely to engage with messages and content they prefer. A user may prefer or respond to one variant of message more frequently than another. This can indicate that a user is more engaged with a certain variant of message related to their regimen than another variant, and thereby is participating more fully in their regimen as a result of receiving that variant. However, some users may not display any obvious or statistically significant preferences, in which case the users can continue to receive variants of messages randomly or with equal frequency from a server.

As these preferences crystalize as indicated from the machine learning model, the service can begin to personalize the user's content by favoring the message types the user reacts to. The service can aggregate the data in the machine learning model (e.g., multi-armed bandit). The more user response data is gathered, the higher the confidence level the model may have in selecting one of the message variants. For a digital therapeutics application, by the time a user completes the session, the service may have updated the model to have the model converge on the user's particular messaging preferences, thereby increasing the likelihood of adherence to the therapeutic interventions provided through the messages.

In this manner, the consumption of computational resources (e.g., processing and memory) of the service and the user device and the network bandwidth can be reduced. By estimating the variant of message preferred by the user using the machine learning model, the server and the user device may reduce the bandwidth, power, or transmissions on messages. Furthermore, the use of the machine learning model may allow for personalization of the message variant selected for the user. By customizing the variants of messages sent to the user within the session to address the user's condition, the quality of HCI between the user and the device is improved. Furthermore, in the context of a digital therapeutics application, the individualized selection of such message variants may result in the delivery of user-specific interventions to improve user's adherence to the treatment. This may result in not only higher adherence to the therapeutic interventions delivered through the session throughout its duration but also potential improvements to the condition of the user to be addressed.

The messages to be delivered may include a number of variants. At least one variant can include an identification of a type or action as part of the session to be performed by a user. In some embodiments, at least one variant for the message may include an identification of a theme of content (e.g., a health, family, work, study) to be presented via the user device. In some embodiments, at least one variant for the message may include an identification of a type of content (e.g., audio, text, image, video, or other multimedia content) to be presented via the user device. In some embodiments, at least one variant may include an identification of inclusion of user information (e.g., inclusion or exclusion of a name of the user on the message when presented on the application). For example, the variant may specify inclusion of a first name of the user, followed by “You have one or more outstanding missions. Tap here to get started!” In some embodiments, at least one variant may specify, identify, or define a layout (e.g., positioning, size, and color) for individual UI elements when the message is presented via the user interface. Each variant of a message can be intended to promote engagement or interaction by the user. Engagement can be measured by a ratio of the messages transmitted to the message interacted with by the user. Selection of which variant of a message can encourage user engagement by selecting a variant which the user is more likely to respond to.

The variants can be based on the user profile or the user. For example, a set of variants may correspond to a personality associated with the user profile. The personality may indicate differences in humor, such as a message which can be in a variant interpreted as “goofy” or “funny” by the user, in contrast to a variant which can be in a variant interpreted as “serious” or “grave” by the user. The variants can be based on a tone of the content included in the message. For example, one variant can be interpreted as empathetic whereas a second variant of the same message can be interpreted as direct. The empathetic variant may say “It is okay to have strong feelings. With this lesson, you'll learn how to better manage them” whereas a direct variant may read “Persistence and self-ownership are the keys to recovery. Log in now to complete your lesson!”

The variants can also be related to the user's knowledge of health or health literacy. The health literacy may correspond to the amount of technical or scientific detail given to the user of the app based on their past experiences. For example, with respect to migraines, variants of the messages may explain to users with health literacy what exactly is happening scientifically in the brain or describe types of symptoms they are having via the application. However, variants of messages for users with lower literacy may include content at a simpler level. For example, a variant can teach a user about an affliction, treatment, or anatomy if the user profile indicates a user has interest or a lack of knowledge in the area. In another example, the variant may say “This medication is increasing your dopamine levels in your brain” as opposed to saying “This medication is making you feel better.” The variants may also be related to a cognitive ability of the user. The cognitive ability can be defined as an ability to do the following: reading, learning, remembering, logical reasoning, and paying attention. The variants 225 may be selected based on the cognitive ability of the user to perform the above tasks and may include different levels of content based on the abilities.

In addition, the variants can be related to a learning style of the user. For example, the user may prefer to learn through visual aids as opposed to text. The variant presented to the user may include graphics or images more conducive towards the user's learning style. The variants can relate to a personal incentive or motivation of the user. For example, the variants may include motivating messages such as “Good Job!” or “Great Work!”, or otherwise providing the user with motivation, such as through sound effects, a point system, or other means. The variants can relate to a preferred messaging style of the user. For example, the user may prefer messages with emoticons, or other images over the message with only plain text lacking emoticons. The variants can relate to a value of the user. For example, a user may place value in their family, or in their studies, and the variant received can correspond to positive reminders of that value, or encouragement based on that value. For example, the message could read “These lessons can help you enjoy being around family and friends again. Start your first lesson:” or “Think about why you're taking these steps. Getting well can help you focus on tasks at home, school, or work. Start your first lesson:” or “Remember that you're doing this for your kids” or “This will help make studying so much easier!” Each variant can be selected based at least on the user profile.

Aspects of the present disclosure are directed to a system, methods, and computer readable media for controlling transmissions of messages to users. A server may transmit, over a first time period of a session for a user towards achieving a behavioral endpoint, one or more first instructions to a user device associated with the user, each of the one or more first instructions to present a message in at least one of one or more variants. A server may receive, from the user device over the first period, one or more responses corresponding to the one or more first instructions, each of the one or more responses by the user to a respective presentation of the message in at least one of the one or more variants towards achieving the behavioral endpoint. A server may determine, responsive to elapsing of the first time period of the session, a respective performance measure of the message in each variant of the one or more variants using the one or more responses. A server may select, for a second time period of the session, a variant from the one or more variants of the message based on the performance measure for the variant. A server may transmit, during the second time period, a second instruction to the user device associated with the user to present the message in the selected variant.

In some embodiments, the server may determine that a second performance measure of the message in the selected variant during the second time period is different from the respective performance metric during the first time period by a threshold margin. The server may select, responsive to determining that the second performance measure is different, a second variant from the plurality of variants for the message to transmit.

In some embodiments, the server may select, for a third time period of the session, a second variant of one or more second variants for a second message to transmit based on the selection of the variant for the message for the second time period. In some embodiments, the server may establish, using the one or more responses, a model to track, for each variant of the one or more variants, (i) the respective performance measure identifying a degree of interactivity by the user with the message in the corresponding variant and (ii) a variance of the performance measure.

In some embodiments, the server may determine to select one of the one or more variants of the message for the second time period, responsive to determining that a variance of the performance measure is below a threshold. In some embodiments, the server may determine, based on a profile of the user, a plurality of prior probabilities of selecting the corresponding plurality of variants of the message for transmission during the first time period of the session.

In some embodiments, the server may provide a dashboard interface for presentation identifying one or more statistics for the session based on the plurality of responses from the user device associated with the user. In some embodiments, determining the respective performance measure further includes determining the respective performance measure of the message in each variant of the one or more variants using the one or more response and a profile of the user identifying information for the session.

In some embodiments, each variant of the one or more variants for the messages can include at least one of: (i) an identification of a type of an action to be performed by the user via the user device, (ii) an identification of a theme for content to be presented via the user device, (iii) an identification of a type of content to be presented via the user device, (iv) an identification of inclusion of user information, (v) a definition of a respective layout for the content, (vi) an identification corresponding to a personality associated with the user, (v) an identification of a tone of the content to be presented via the user device, (vi) an identification of content based on the literacy of the user, (vii) an identification of content based on the health knowledge of the user, (viii) an identification a learning style associated with the user, (ix) an identification of a personal incentive or motivation of the user, (x) an identification of a messaging style of the user, (xi) an identification of values of the user, or (xii) a cognitive ability of the user.

In some embodiments, the server may maintain, on a database, a profile of the user identifying (i) the behavioral endpoint to be achieved, (ii) a log record for the one or more responses from the user, and (ii) one or more performance metrics for the one or more variants of the message. In some embodiments, the user may be on a medication associated with addressing the behavioral endpoint, at least in partial concurrence with the session.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts a block diagram of a system for controlling transmissions of messages to user devices in accordance with an illustrative embodiment;

FIG. 2 depicts a block diagram for a process to select and transmit variants of messages during a first time period of a session in the system for controlling transmissions in accordance with an illustrative embodiment;

FIG. 3 depicts a block diagram for a process to receive responses to messages during a first time period of a session in the system for controlling transmissions in accordance with an illustrative embodiment;

FIG. 4 depicts a block diagram for a process to select and transmit variants of messages during a second time period of a session in the system for controlling transmissions in accordance with an illustrative embodiment;

FIG. 5 depicts a set of example graphs for prior probabilities of message variants in accordance with an illustrative embodiment;

FIGS. 6A-B each depict a method for predicting variants of a message to transmit to a user device based on the user device's responses in accordance with an illustrative embodiment; and

FIG. 7 is a block diagram of a server system and a client computer system in accordance with an illustrative embodiment.

### DETAILED DESCRIPTION

For purposes of reading the description of the various embodiments below, the following enumeration of the sections of the specification and their respective contents may be helpful:

Section A describes systems and methods for adaptive selection of messages for session adherence; and

Section B describes a network and computing environment which may be useful for practicing embodiments described herein.

### A. Systems and Methods for Adaptive Selection of Messages for Session Adherence

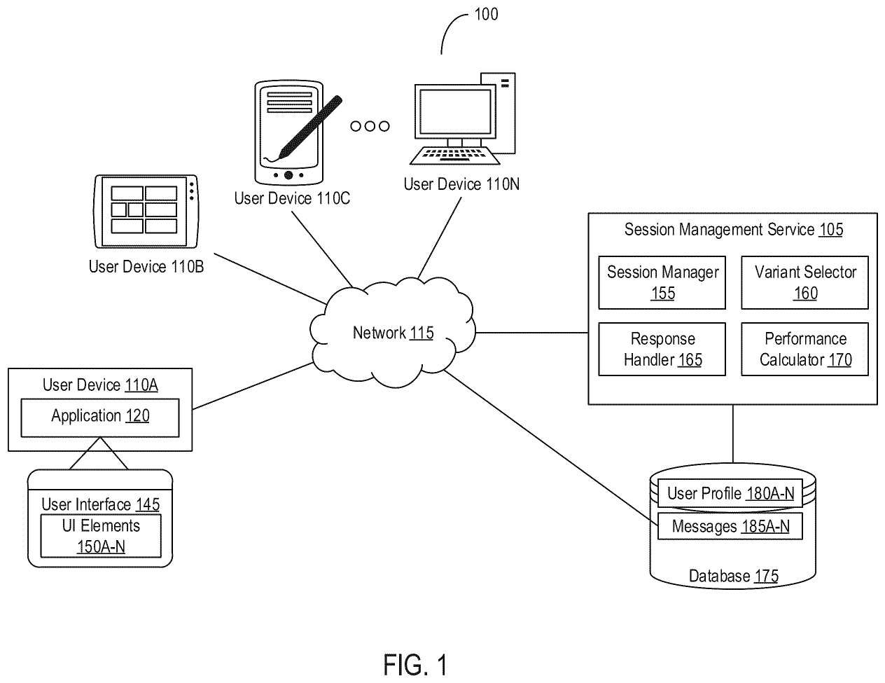

Referring now to FIG. 1, depicted is a block diagram of a system 100 for controlling transmissions of messages to users. In an overview, the system 100 may include at least one session management service 105 and a set of user devices 110A-N (hereinafter generally referred to as user devices 110), communicatively coupled with one another via at least one network 115. At least one user device 110 (e.g., the first user device 110A as depicted) may include at least one application 120. The application 120 may include or provide at least one user interface 145 with one or more user interface (UI) elements 150A-N (hereinafter generally referred to as UI elements 150). The session management service 105 may include at least one session manager 155, one variant selector 160, one response handler 165 or at least one performance calculator 170, among others. The session management service 105 may include or have access to at least one database 175. The database 175 may store, maintain, or otherwise include one or more user profiles 180A-N (hereinafter generally referred to as user profiles 180) and one or more messages 185A-N among others. The functionality of the application 120 may be performed in part on the session management service 105.

In further detail, the session management service 105 may (sometimes herein generally referred to as a computing system or a service) be any computing device comprising one or more processors coupled with memory and software and capable of performing the various processes and tasks described herein. The session management service 105 may be in communication with the one or more user devices 110 and the database 175 via the network 115. The session management service 105 may be situated, located, or otherwise associated with at least one server group. The server group may correspond to a data center, a branch office, or a site at which one or more servers corresponding to the session management service 105 is situated.

Within the session management service 105, the session manager 155 may execute, initiate, or send messages 185 related to a session initiated by a user of the application 120 on respective user devices 110. The variant selector 160 may select a variant of a message to transmit via the session manager to a user device 110. The response handler 165 may receive, from the user device 110, responses to messages. The performance calculator 170 may calculate or update a model of responses and messages.

The user device 110 (sometimes herein referred to as an end user computing device) may be any computing device comprising one or more processors coupled with memory and software and capable of performing the various processes and tasks described herein. The user device 110 may be in communication with the session management service 105 and the database 175 via the network 115. The user device 110 may be a smartphone, other mobile phone, tablet computer, wearable computing device (e.g., smart watch, eyeglasses), or laptop computer. The user device 110 may be used to access the application 120. In some embodiments, the application 120 may be downloaded and installed on the user device 110 (e.g., via a digital distribution platform). In some embodiments, the application 120 may be a web application with resources accessible via the network 115.

The application 120 executing on the user device 110 may be a digital therapeutics application and may provide a session (sometimes referred to herein as a therapy session) to address at least one condition of the user. The condition of the user may include, for example, a chronic pain (e.g., associated with or include arthritis, migraine, fibromyalgia, back pain, Lyme disease, endometriosis, repetitive stress injuries, irritable bowel syndrome, inflammatory bowel disease, and cancer pain), a skin pathology (e.g., atopic dermatitis, psoriasis, dermatillomania, and eczema), a cognitive impairment (e.g., mild cognitive impairment (MCI), Alzheimer's, multiple sclerosis, and schizophrenia), and other ailments (e.g., narcolepsy and oncology), among others.

The user may be at least partially concurrently taking medication to address the condition, while being provided sessions through application 120. For instance, if the medication is for pain, the user may be taking: acetaminophen; a nonsteroidal anti-inflammatory composition; an antidepressant; an anticonvulsant; or other composition; among others. For skin pathologies, the user may be taking: a steroid, antihistamine, or topic antiseptic, among others. For cognitive impairments, the user may be taking cholinesterase inhibitors or memantine, among others. For narcolepsy, the user may be taking a stimulant or antidepressant, among others. The user of the application 120 may also other psychotherapies for these conditions.

The application 120 can include, present, or otherwise provide a user interface 145 including the one or more UI elements 150 to a user of the user device 110 in accordance with a configuration on the application 120. The UI elements 150 may correspond to visual components of the user interface 145, such as a command button, a text box, a check box, a radio button, a menu item, and a slider, among others. In some embodiments, the application 120 may be a digital therapeutics application and may provide a session (sometimes referred to herein as a therapy session) via the user interface 145 towards achieving a behavioral endpoint of the user (sometimes herein referred to as a patient, person, or subject). A behavioral endpoint can be, for example a completion of the session, a physical or mental goal of a user, a completion of a medication regimen, or a behavioral endpoint indicated by a doctor or a user.

The database 175 may store and maintain various resources and data associated with the session management service 105 and the application 120. The database 175 may include a database management system (DBMS) to arrange and organize the data maintained thereon. The database 175 may be in communication with the session management service 105 and the one or more user devices 110 via the network 115. While running various operations, the session management service 105 and the application 120 may access the database 175 to retrieve identified data therefrom. The session management service 105 and the application 120 may also write data onto the database 175 from running such operations.

Such operations may include transmittal and receipt of messages 185. The message 185 can be an MMS, SMS, or via a messaging interface of the application 120. Each message 185 may identify or include information to be presented via the user interface 145 of the application 120. For example, the information of message 185 may include reminders to perform a task of the session. The message 185 may be delivered periodically, such as daily, weekly, monthly, etc. The message 185 may be derived from a library of pre-generated psychotherapy messages and/or a library of pre-generated engagement (remind) messages. The message 185 may include reminders for the subject to complete the therapy sessions, to take medication, and/or to complete a task of the regimen. The message 185 may be personalized based on the user's activity, adherence, and/or performance in relation to the regimen.

The message 185 may also include a mechanism for responding, such as a link, chat box, or indication to respond to the message. The session manager 155 can coordinate the transmittal of messages 185 to the user device 110. The response handler 165 can coordinate the receipt of responses to the messages 185 and subsequently update a user profile 180 responsive to the user's responses.

The user profile 180 (sometimes herein referred to as a user account, user information, or subject profile) can store and maintain information related to a user of the application 120 through user device 110A. Each user profile 180 may be associated with or correspond to a respective user 210 (described herein) of the application 120. The user profile 180 may identify various information about the user 210, such as a user identifier, the condition to be addressed, information on sessions carried out by the user 210, message preferences, user demographic information, and progress in addressing the condition, among others. The information on a session may include various parameters of previous sessions performed by the user 210, and may be initially null. Message preferences can include treatment preferences and user input preferences, such as types of messages or timing of messages preferred. Message preferences can also include preferences determined by the session management service 105, such as a type of or variant of message the user 210 may respond to. The progress may initially be set to a start value (e.g., null or “0”) and may correspond to alleviation, relief, or treatment of the condition.

In some embodiments, the user profile 180 may identify or include information on a treatment regimen undertaken by the user 210, such as a type of treatment (e.g., therapy, pharmaceutical, or psychotherapy), duration (e.g., days, weeks, or years), and frequency (e.g., daily, weekly, quarterly, annually), among others. The user profile 180 may be stored and maintained in the database 175 using one or more files (e.g., extensible markup language (XML), comma-separated values (CSV) delimited text files, or a structured query language (SQL) file). The user profile 180 may be iteratively updated as the user 210 performs additional session or responds to additional messages 185. For example, the response handler 165 may update the user profile 180 based on a response provided by the user. A response provided by the user can indicate an engagement with a message 185, a change in demographic information, or an update of user preferences.

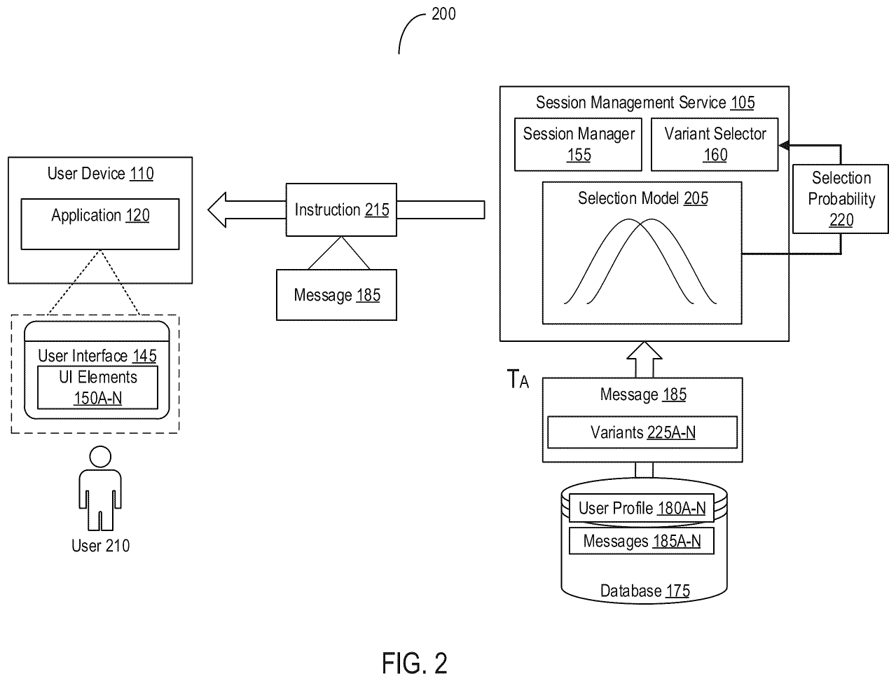

Referring now to FIG. 2, depicted is a block diagram for a process 200 to select and transmit variants of messages during a first time period of a session in the system for controlling transmissions. The process 200 may include or correspond to operations performed in the system 100 to select and transmit variants of messages for a first time period of a session (during time T_{A}). Under the process 200, the session manager 155 executing on the session management service 105 may access the database 175 to retrieve or identify the user profile 180 for a user 210 of the application 120 on the user device 110. From the user profile 180, the session manager 155 may determine or identify a behavioral endpoint for the user 210.

The session manager 155 may determine or identify a session for the user 210 towards achieving the behavioral endpoint. The application 120 executing on the user device 110 may be a digital therapeutics application and may provide a session (sometimes referred to herein as a therapy session) via the user interface 145 to address at least one condition of the user 210 (sometimes herein referred to as a patient, person, or subject). The session provided via the application 120 may include a set of messages with routines or activities to be performed by the user 210 or information to be read or viewed by the user 210. The session may identify or have a set duration of time for the condition to be addressed. The duration of time may have any range of time, such as from a few hours (e.g., 2 hours) to multiple years (e.g., 3 years), among others. The prompt of the message presented on the user device 110 by the session management service 105 on a user interface 145.

To start a session, the application 120 on the user device 110 may provide, send, or otherwise transmit at least one request for the session for the user 210 of the application 120 to the session management service 105. The session may correspond to a set of routines or activities the user 210 is to carry out and be evaluated on. The request may include or identify the user 210 to which the session is to be provided. In some embodiments, the application 120 may send the request in response to detecting a corresponding interaction with the interface 145 of the application 120. In some embodiments, the application 120 may send the request independent of interaction by the user 210 with the interface 145, for example, in accordance with a schedule for addressing the condition of the user 210. The session manager 155 can, responsive to receiving the request, initiate a session.

With the identification of the user profile 180, the session manager 155 may select or identify a set of messages 185 for the session. For example, the database 175 may have multiple sets of messages 185 for different conditions. The session manager 155 may select the set of messages 185 for the condition of the user 210 as identified in the user profile 180. The set of messages 185 may define or form the session for the user 210. Each message 185 may identify or have a set of variants 225A-N. Each variant 225 of the message 185 may convey or include at least a common portion of information of the message 185 across all the variants 225. For example, a first variant 225A may express the same prompt to perform a particular action of the same routine as a second variant 225B, but with different formatting (e.g., layout), content type (e.g., text, audio, or video), or theme (e.g., in a different tone).

In some embodiments, at least one variant 225 can include an identification of a type or action as part of the session to be performed by a user 210. In some embodiments, at least one variant 225 for the message 185 may include an identification of a theme of content (e.g., a health, family, work, study) to be presented via the user device 110. In some embodiments, at least one variant 225 for the message 185 may include an identification of a type of content (e.g., audio, text, image, video, or other multimedia content) to be presented via the user device 110. In some embodiments, at least one variant 225 may include an identification of inclusion of user information (e.g., inclusion or exclusion of a name of the user on the message 185 when presented on the application 120). For example, the variant 225 may specify inclusion of a first name of the user, followed by “You have one or more outstanding missions. Tap here to get started!” In some embodiments, at least one variant 225 may specify, identify, or define a layout (e.g., positioning, size, and color) for individual UI elements 150 when the message 185 is presented via the user interface 145. Each variant of a message can be intended to promote engagement or interaction by the user 210. Engagement can be measured by a ratio of the messages 185 transmitted to the message 185 interacted with by the user. Selection of which variant 225 of a message 185 can encourage user engagement by selecting a variant 225 which the user 210 is more likely to respond to.

The message 185 may include or contain multiple variants 225. The variants 225 can be based on the user profile 180 or the user 210. For example, a set of variants 225 may correspond to a personality associated with the user profile 185. The personality may indicate differences in humor, such as a message 185 which can be in a variant interpreted as “goofy” or “funny” by the user 210, in contrast to a variant which can be in a variant interpreted as “serious” or “grave” by the user 210. The variants 225 can be based on a tone of the content included in the message 185. For example, one variant 225 can be interpreted as empathetic whereas a second variant 225 of the same message 185 can be interpreted as direct. The empathetic variant 225 may say “It is okay to have strong feelings. With this lesson, you'll learn how to better manage them” whereas a direct variant 225 may read “Persistence and self-ownership are the keys to recovery. Log in now to complete your lesson!” The tone of the content may be identified in the variant 225 for the message 185.

The variants 225 can be related to the user's 210 knowledge of health or health literacy. The health literacy may correspond to the amount of technical or scientific detail given to the user of the app based on their past experiences. For example, with respect to migraines, variants 225 may explain to users with health literacy what exactly is happening scientifically in the brain or describe types of symptoms they are having via the application. However, variants 225 for users with lower literacy may include content at a simpler level. For example, a variant 225 can teach a user 210 about an affliction, treatment, or anatomy if the user profile 180 indicates a user 210 has interest or a lack of knowledge in the area. For example, the variant 225 may say “This medication is increasing your dopamine levels in your brain” as opposed to saying “This medication is making you feel better.” The health knowledge or literacy may be identified in the variant 225 for the message 185. The variants 225 may also be related to a cognitive ability of the user 210. The cognitive ability can be defined as an ability to do the following: reading, learning, remembering, logical reasoning, and paying attention. The variants 225 may be selected based on the cognitive ability of the user to perform the above tasks and may include different levels of content based on the abilities.

In addition, the variants 225 can be related to a learning style of the user 210. For example, the user 210 may prefer to learn through visual aids as opposed to text. The variant 225 presented to the user 210 may include graphics or images more conducive towards the user's learning style. The learning style of the user may be identified in the variant 225. The variants 225 can relate to a personal incentive or motivation of the user 210. For example, the variants 225 may include motivating messages such as “Good Job!” or “Great Work!”, or otherwise providing the user 210 with motivation, such as through sound effects, a point system, or other means. The personal incentive or motivation of the user 210 may be identified in the variant 225 for the message 185. The variants 225 can relate to a preferred messaging style of the user 210. For example, the user 210 may prefer messages 185 with emoticons, or other images over the message 185 with only plain text lacking emoticons. The messaging style may be identified in the variant 225 of the message 185. The variants 225 can relate to a value of the user 210. For example, a user may place value in their family, or in their studies, and the variant 225 received can correspond to positive reminders of that value, or encouragement based on that value. The values of the user 210 may be identified in the variant 225 for the message 285. For example, the message could read “These lessons can help you enjoy being around family and friends again. Start your first lesson:” or “Think about why you're taking these steps. Getting well can help you focus on tasks at home, school, or work. Start your first lesson:” or “Remember that you're doing this for your kids” or “This will help make studying so much easier!” Each variant 225 can be selected by the variant selector 160 based at least on the user profile 180.

The variant selector 160 executing on the session management service 105 may initiate or establish at least one selection model 205 for controlling and selecting the variants 225 of the message 185 to provide to the user 210 associated with the user device 110. The selection model 205 (sometimes herein referred to as a machine learning model or a statistical model) may iteratively develop over a first time period T_{A} in which data is collected by the session management service 105. Collection of data over a time period T_{A} can facilitate the development of an accurate and robust selection model 205. In some instances, collecting data over a brief period of time or collecting an insignificant amount of data can lead to an inaccurate model. Therefore, during a specified period of time (T_{A}) or up until a specified number of responses, enough responses to variants can be recorded for the variant selector 160 to establish a selection model 205. The selection model 205 can be based on various models or algorithms, such a statistical model (e.g., multi-armed bandit or multiclass queueing network) or reinforcement model (e.g., Q-learning or Markov decision process), among others. In general, the selection model 205 may be used to track a selection probability for each variant 225, a performance measure for each variant 225, and a statistical variance of the performance for each variant 225, among others.

For example, the selection model 205 may be implemented using a multi-armed bandit model updated using Thompson sampling. The multi-armed bandit model can be a probability heuristic in which multiple resources have an unknown return probability associated with various payoffs and in which it is desirable to maximize the payoff. Furthermore, each resource can provide a random payoff dependent upon an unknown probability distribution of the given resource. The multi-armed bandit model can seek to develop a metric for the payoff (e.g., gain) of each resource as well as the probability distribution of the payoff for each resource through iterative selection of each resource over a period of time to converge on a maximized payoff. The multi-armed bandit model can be represented as

$$ρ = T {μ}^{*} {\sum }_{t = 1}^{T} {\hat{r}}_{t}$$

where μ* is the maximal payoff mean (e.g., if a user were to interact with every variant), ρ is the regret, T is the number of rounds (e.g., message variants sent), and \hat{r}ₜ is the payoff (e.g., a user interaction with the variant, no user interaction with the message variant, or other performance measure) in round t.

Thompson sampling can define a heuristic for choosing variants demonstrated in the multi-armed bandit model. The sampling may include a model for choosing a variant which can be expected to maximize payoff (e.g., user responses or performance measure). Thompson sampling may include on several elements, comprising: (1) A likelihood function: P (r|Θ, a, x); (2) a set Θ of parameters θ of the distribution of r; (3) a prior distribution P (θ); (4) a past observations triplet D={(x; a; r)}; and (5) a posterior distribution P (θ|D), wherein x refers to a context (e.g., a user presented the message), a is an action (e.g., a user receiving a variant of a message), and r is a reward (e.g., payoff or the user interacting with the variant). Thompson sampling can rely on various rounds of sampling (e.g., multiple message rounds to a user in different variants) to maximize the expected result over time or rounds.

The selection model 205 can be updated and developed in conjunction with the heuristics detailed herein, or with other probability models. The selection model 205 can be developed from an iterative sampling of messages sent to user. This iterative sampling can, in an implementation, be used to develop probabilities of the success (e.g., illiciting a response from the user) of each variant 225 of message 185. For example, the variant selector 160 can initially assign an equal probability of success (e.g., a selection probability 220) of each variant 225 of a message 185. This prior probability (e.g., selection probability 220) can be iteratively updated as new information pertaining to the selection probability 220 is obtained. For example, a response to a message 185, user selected preferences, or a user's demographic can effect or change a selection probability 220 of a variant 225. Selection probability 220 can vary or change from its initial equalized probability as more information is obtained. In this manner, a probability distribution of the likelihood of success of each variant 225 of a message 185 can be converged upon.

The user's response pattern can create a statistical variance. For example, a user may not exhibit any predictable behavior of variant 225 preference. This can result in a large variance of variant preference. In this instance, variant 225 of a message 185 can be sent randomly, or with equal frequency. In another example, a user 210 may respond to a variant 225 of a message 185 in a manner inconsistent with the developed selection model 205 or prior probabilities associated with the variant 225 (stored in the selection model 205). This can create a statistical variance within a probability distribution of success of a variant 225. A variance can be the expectation of the squared deviation of a variant 225 from its mean. In some instances, the selection model 205 can continue to collect message response data from a user 210 over a time period T_{A} until the variance among the variants 225 is below a threshold value. The variant selector 160 can include other information beyond user 210 responses to messages 185 in order to develop the selection model 205.

In some embodiments, the selection model 205 can leverage information contained in the database such as the user profiles 180 to determine probabilities of message variants. The user profiles 180 can include demographic information of a user 210, such as age, weight, or health conditions, among others. Demographic information can be provided by the user 210 (e.g., via the user interface 145) or can be gathered about the user, and may identify various traits about the user 210. For example, the session management service 105 can identify that a user 210 has begun a session corresponding to managing pre-diabetes, and can update the user profile 180 to conclude that the user 210 has pre-diabetes. The demographic information about a user can affect the selection probability 220 of the variant 225.

In some embodiments, the variant selector 160 may calculate, generate, or determine a prior probability of selection in the selection model 205 for each variant 225 of the message 185 using the user profile 180. For instance, the variant selector 160 can create subgroups of users with similar demographics using the user profiles 180. The variant selector 160 can use these subgroups to generate more accurate selection probabilities for a group of users. For example, the variant selector 160 can define groupings related to particular traits of the user (e.g., weight, height, health conditions, or any combinations thereof) to determine commonalities among preferred variants 225 of a group. With the definition, the variant selector 160 may identify the selection models 205 for the variant 225 across the group of users. The variant selector 160 may establish the selection model 205 for the user 210 based on a combination of the selection models 205 for the variant 225 across the group of users. The combined selection model 205 may have prior selection probabilities biased toward at least one variant 225, based on the selection model 205 from the other users in the same group. In this manner, the variant selector 160 may present variants 225 which may be more likely to result in a user 210 engagement with the message 185.

The variant selector 160 can identify or otherwise select at least one variant 225 from a set of variants 225 of message 185 to transmit. The selection may be in accordance with the selection model 205 during a first time period of the session (e.g., during time T_{A}). For each variant 225, the variant selector 160 may calculate, determine, or identify a corresponding selection probability 220, as described herein. The variant selector 160 can continuously update the selection model 205, or can update the selection model 205 periodically, or in response to new data (e.g., a user response, change in user demographics). The variant selector 160 can select the variant 225 of message 185 to send according to the variant 225 possessing the highest selection probability 220. In this manner, a variant 225 can be presented to the user 210 (via the application 120 on user device 110) which has a high likelihood of prompting user engagement.

With the selection of a variant 225, the session manager 155 may generate at least one instruction 215 to transmit to the user device 110. The instruction can include the selected variant 225 of the message 185. The instruction 215 may be code, data packets, or a control to present a message to the user 210 in at least one of the variants 225. The instruction 215 may include processing instructions for display of the message on the application 120. The instruction 215 may also include instructions for the user 210 to perform in relation to their session. For example, the instruction 215 may display a message 185 instructing the user to take a medication associated with their session. Upon generation, the session manager 155 can send the instruction 215 to the user device 110.

The application 120 on the user device 110 may retrieve, identify, or otherwise receive the instruction 215 identifying the selected variant 225 of the message 185 from the session management service 105. Upon receipt, the application 120 may display the message 185 via the user interface 145 through application 120. The user interface 145 can display a variant 225A of a message 185A differently than a variant 225B of the same message 185A. For example, a variant 225 can refer to a different display or presentation of the same message. For example, a variant 225 can be a different layout for a UI element 150 displaying on the user interface 145. The message 185 can include or display prompts, buttons, text boxes, links (e.g., URLs), or other means for the user 210 to interact with or engage with the message 185. For example, a presentation of the message 185 can include a button which asserts that a user 210 has performed a task of their session. Engaging with a prompt of the message 185 can be an interaction 305. An interaction 305 can indicate that a user 210 has engaged with a variant 225 of a message 185.

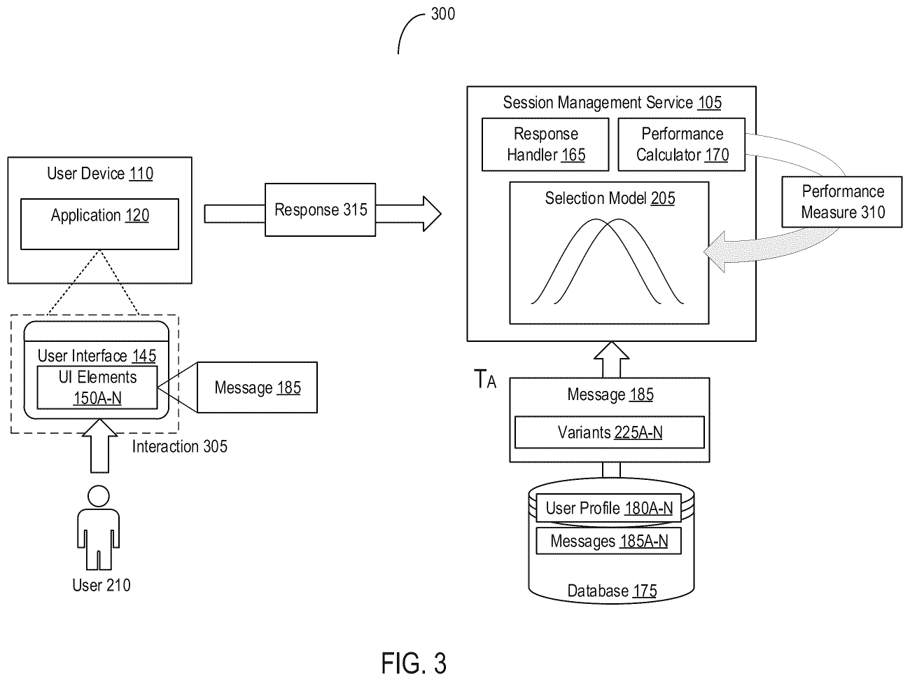

Referring now to FIG. 3, depicted is a block diagram for a process 300 to receive responses to messages during a first time period of a session in the system for controlling transmissions. The process 300 may include or correspond to operations to gather user responses in connection with presentation of variants of messages during the first time period of the session (e.g., during time T_{A}). Under the process 300, the application 120 running on the user device 110 may monitor for at least one interaction 305 with the user interface 145. The interaction 305 may include a keypress, a touch screen, a click, or any other event associated with the presentation of the message 185 via the user interface 145. In some embodiments, the interaction 305 may be subsequent to the presentation of the variant 225 of the message 185. For instance, the interaction 305 may correspond to a set of user interactions to log a completion of a specified routine, after presentation of the variant 225 of the message 185 prompting the user 210 to perform the routine. In some embodiments, the interaction 305 may be concurrent with the presentation of the message 185 via the user interface 145.

Upon detection of the interaction 305, the application 120 may produce or generate at least one response 315 for transmission to the session management service 105. The response 315 may identify or include information pertaining to a user completion of a task, a response by the user 210 or general engagement with the message 185 or session. In some embodiments, the application 120 may maintain a timer to keep track of time elapsed since presentation of the message 185. The application 120 may compare the elapsed time with a time limit for the variant 225 of the message 185. When the elapsed time exceeds the time limit, the application 120 may generate the response 315 to indicate no user interaction with the variant 225 of the message 185. Upon generation, the application 120 may provide, transmit, or otherwise send the response 315 to the session management service 105.

The response handler 165 executing on the session management service 105 may retrieve, identify, or otherwise receive the response 315 from the user device 110. Upon receipt, the response handler 165 may parse the response 315 to extract or identify the interaction 305 of the user 210 with the presented variant 225 of the message 185. The response handler 165 may record or log the data from the response 315 into the database 175. The response handler 165 may store an association between the response 315 and the user profile 180. In some embodiments, the response handler 165 may store and maintain identifications of the variants 225 of the session in the database 175. The response handler 165 may store an association between the response 315 and the variant 225 for the user 210.

The performance calculator 170 executing on the session management service 105 can calculate, generate, or otherwise determine a performance measure 310 for the variant 225 of the message 185. The performance measure 310 can be a value (e.g., numeric value) identifying or corresponding to the interaction 305 of the user 210 with the presented variant 225 of the message 185 on the user interface 145. The performance measure 310 may be assigned or set to one value when the presented variant 225 elicits the interaction 305 and another value when the variant 225 does not elicit an interaction 305. In general, more interactions with the variant 225 of the message 185 may result in a higher performance measure 310, whereas less or no interactions with the variant 225 of the message 185 when presented may result in a lower performance measure 310. In some embodiments, the performance measure 310 may correspond to a rate of correct or proper responses by the user 210 to the variant 225 of the message 185. The rate of correct or proper responses may be correlated with adherence to the session for addressing the condition. The performance calculator 170 can analyze information pertaining to the responses 315 and the variants 225 and can perform statistical calculations. For example, the performance calculator can calculate variances of frequency of responses 315, relationships between a specific variant 225 and a specific response 315 rate, or between a user profile 180 and a variant 225.

In some embodiments, the performance measure 310 may correlate to the variance of the responses 315 as associated with the variants 225 for a given user 210. For example, the performance calculator 170 may calculate a variance between variants 225 which have prompted interactions 305 from a user 210 and variants 225 which have not prompted an interaction 305 from the user 210. The performance calculator 170 may also calculate the performance measure 310 of each variant 225 of the message 185 using the response 315 gathered over time T_{A} and the user profile 180 corresponding to the responses 315. In this manner, the performance measure 310 can relate to a degree of interactivity by the user 210 with the message 185 in the corresponding variant 225.

Using the performance measure 310, the performance calculator 170 may modify or update the selection model 205. As discussed above, the selection model 205 may keep track of the selection probability for each variant 225, the performance measure for each variant 225, and the statistical variance of the performance for each variant 225, among others. The performance calculator 170 may update the selection model 205 to change the selection probability 220 of the variant 225A based on the performance measure 310. For example, if a user 210 consistently does not respond to the variant 225A, the performance calculator 170 may assign a lower selection probability 220 to the presented variant 225. Conversely, if the user 210 responded to the variant 225, the performance calculator 170 may assign a higher selection probability 220. In either scenario, with the addition of responses 315, the performance calculator 170 may reduce the statistical variation for the variant 225 in the selection model 205.

The performance calculator 170 may modify or update the user profile 180 to identify or include the performance measure 310, selection probabilities 220, or other statistics associated with the user 210 represented by user profile 180. The performance calculator 170 may maintain information pertaining to the user 210 on the user profile 180 in the database 175. The user profile 180 may identify the behavioral endpoint of the user, a log record of the responses 315 associated with the user, or the set of performance measures 310 associated with the user 210 for the variants 225 presented to the user 210. The performance calculator 170 may update the selection model 205 based on information within the user profile 180, the performance measure 310, or a combination thereof.

With the updating of the selection model 205, the performance calculator 170 may determine whether the first time period T_{A} for the session has elapsed. The time period T_{A} may correspond to a portion of the session during which any variant 225 of the message 185 may be selected for transmission to the user 210. During this time, the session management service 105 may continue to collect responses 315 from the user device 110 to be used to select one of the variants 225 of the message 185 to provide for at least a portion of the remainder of the session. The elapsing of the time period T_{A} may correspond to at least one variance in the selection model 205 being below a threshold, a threshold number of responses 315 received by the session management service 105, or a set time limit, among others.

In some embodiments, the performance calculator 170 may determine whether the time limit for the first time period of the session has elapsed. The time limit may be defined relative to a start of the session, and may range between a few seconds (e.g., 3 seconds) to a few weeks (e.g., 10 weeks), among others. When the time limit has elapsed, the performance calculator 170 may determine that the first time period T_{A} for the session has elapsed. Otherwise, when the time limit has yet to elapse in the session, the performance calculator 170 may determine that the first time period T_{A} for the session has not elapsed.

In some embodiments, the performance calculator 170 may determine whether at least one statistical variance for the corresponding variant 225 of the message 185 in the selection model 205 is below a threshold. The threshold may delineate or identify a value for the statistical variance at which sufficient confidence is attained for selection of the variant 225 for at least a portion of the remainder of the session. When the variance is less than the threshold, the performance calculator 170 may determine that the first time period T_{A} for the session has elapsed. Otherwise, when the variance is more than the threshold, the performance calculator 170 may determine that the first time period T_{A} for the session has not elapsed. The session management service 105 may then repeat the processes 200 and 300 as discussed herein.

In some embodiments, the performance calculator 170 may determine whether a number of responses 315 from the user 210 is greater than a threshold. The threshold may delineate or identify a value for the number of responses 315 at which sufficient confidence is attained for selection of the variant 225 for at least a portion of the remainder of the session. When the number of responses is greater than or equal to the threshold, the performance calculator 170 may determine that the first time period T_{A} for the session has elapsed. Otherwise, when the number of responses is less than the threshold, the performance calculator 170 may determine that the first time period T_{A} for the session has not elapsed. The session management service 105 may then repeat the processes 200 and 300 as discussed herein.

Subsequent to the determination of the first time period elapsing, the second time period T_{B} for the session may begin. In some embodiments, subsequent to the first time period elapsing, the session management service 105 can stop logging responses, calculating variances, or other operations described in conjunction with T_{A}. In some embodiments, the first time period elapsing can indicate that sufficient data has been captured in the selection model 205 to make a variant 225 selection by the session management service 105 which is likely to elicit a response 315 from the user 210.

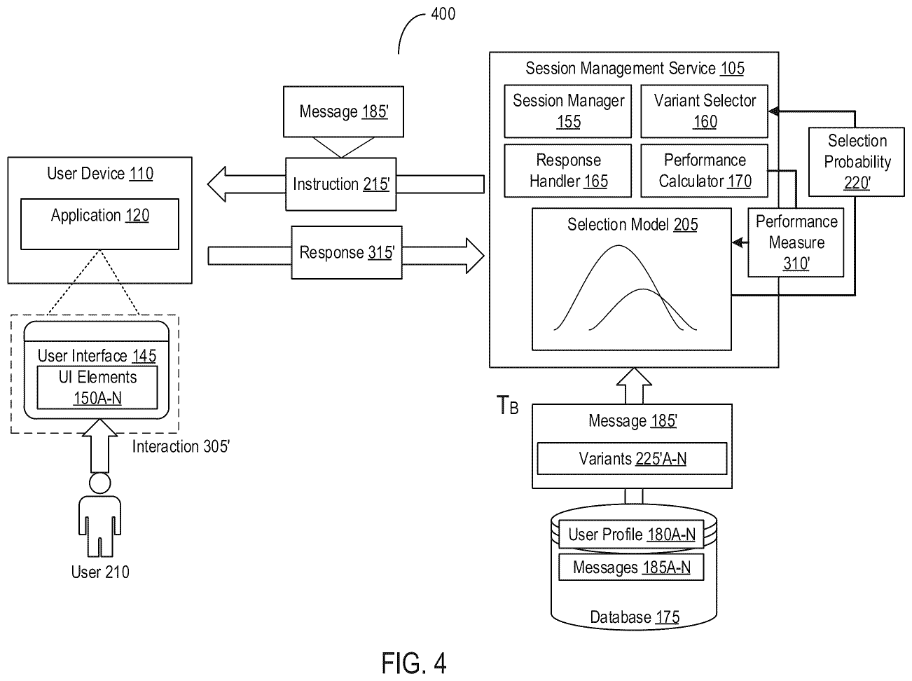

Referring now to FIG. 4, depicted is a block diagram for a process 400 to select and transmit variants of messages during a second time period of a session in the system for controlling transmissions. The process 400 can include operations corresponding to transmission of messages and receipt of responses during a second period of time for a session (during time T_{B}). The process 400 may include one or more operations discussed in conjunction with processes 200 or 300. Under the process 400, the session manager 155 may continue with the session (now in the second period of time). In the second period of time for the session, the session manager 155 may identify or select at least one message 185′ to transmit to the user 210. In some embodiments, the message 185′ may be the same as the message 185 from the first period of time in the session. In some embodiments, the message 185′ may be different from the message 185 transmitted during the first period of the session.

In some embodiments, the session manager 155 may identify or select another set of messages 185′ for at least a portion of the second period of time for the session to send to the user 210. Subsequent to the first time period T_{A} in which the selection model 205 is initially established, the session manager 155 can identify or select the second set of message 185′. The second messages 185′ may be for the same session established to address the condition of the user 210. The second set of messages 185′ may have a relation with the first set of messages 185 used in the first period of time. The relation between the sets of messages may be defined in accordance with the specifications of the session. For example, the second set of messages 185′ can be for a subsequent routine or activity prompted by the message 185 in time period T_{A}. In this example, the messages in T_{A} can relate to performing a breathing exercise, and the message in T_{B} can relate to taking a running exercise.

Each message 185′ may also have a set of variants 225′A-N, similar to the set of variants 225 as discussed above. Each variant 225′ of the message 185′ may convey or include at least a common portion of information of the message 185′ across all the variants 225′, with different action type, theme, content type, inclusion of user information (e.g., name), or layout, among others. Each of variants 225′ of the message 185′ may have a correspondence with the one of variants 225 of the message 185. For instance, the variant 225′ and variant 225 may have correspondence with the same type of action, theme, content type, whether to include user information, or layout, among others.

With the identification, the variant selector 160 may identify or select a variant 225′ of the message 185′ using the selection model 205. The variant selector 160 may select the variant 225′ of the message 185′ determined to have highest selection probability 220′ from the first time period for the session. In some embodiments, the variant selector 160 may select the variant 225 of the message 185′ different from the message 185 selected during the first time period using the selection model 205. To select, the variant selector 160 may identify the variant 225 with the highest selection probability 220′ from the selection model 205. With the identification, the variant selector 160 may find, determine, or otherwise select a variant 225′ for the message 185′ with the correspondence with variant 225 for the message 185. For example, the variant selector 160 may select the variant 225′ for the message 185′ with the same layout as the variant 225 for the message 185.

In some embodiments, the variant selector 160 may identify or select a variant 225′ of the message 185′ different from the highest selection probability 220 as determined using the selection model 205 from the first time period. The selection probability 220′ of the performance measure 310 of a variant 225 can change from the first time period T_{A} to the second time period T_{B}. This change can be due to a variety of reasons, such as a change in the user 210 preferences, a change in the user's condition or environment, a change in the one or more variants 225 (such as additional variants 225 being added or removed), a change in the user device 110 that would cause a different presentation of the variant 225 to the user (e.g., a user buying a different mobile device to display the application 120 on), among other factors.

To determine, the variant selector 160 may determine that the selection probability 220′ in the second time period differs from the selection probability 220 during the first time period by a threshold margin. The threshold margin may identify or define a value for the difference in the selection probability 220 and selection probability 220′ at which the preference of the user 210 has deviated. In some embodiments, the performance measure from the user 210 may be used instead of or in addition to the selection probabilities. When the difference is greater than the threshold margin, the variant selector 160 can select a different variant 225′ different from the initially selected variant 225. Otherwise, when the difference is lower than the threshold margin, the variant selector 160 may maintain the initially selected variant 225.

With the selection, the session manager 155 may generate at least one instruction 215′ to transmit to the user device 110. The instruction can include the selected variant 225′ of the message 185′. The instruction 215′ may be code, data packets, or a control to present a message to the user 210 in at least one of the variants 225′. The instruction 215′ may include processing instructions for display of the message on the application 120. The instruction 215′ may also include instructions for the user 210 to perform in relation to their session. For example, the instruction 215′ may display the message 185′ instructing the user to perform a certain activity associated with their session. Upon generation, the session manager 155 can send the instruction 215′ to the user device 110.

The application 120 on the user device 110 may retrieve, identify, or otherwise receive the instruction 215′ identifying the selected variant 225′ of the message 185′ from the session management service 105. Upon receipt, the application 120 may display the message 185′ via the user interface 145 through application 120. With presentation, the application 120 may monitor for at least one interaction 305′ with the user interface 145. In some embodiments, the interaction may be subsequent to the presentation of the variant 225′ of the message 185′. For instance, the interaction 305′ may correspond to a set of user interactions to log a completion of a specified routine, after presentation of the variant 225′ of the message 185′ prompting the user 210 to perform the routine. In some embodiments, the interaction 305′ may be concurrent with the presentation of the message 185′ via the user interface 145.

Upon detection of the interaction 305′, the application 120 may produce or generate at least one response 315′ for transmission to the session management service 105. The response 315′ may identify or include information pertaining to a user completion of a task, a response by the user 210 or general engagement with the message 185′ or session. In some embodiments, the application 120 may maintain a timer to keep track of time elapsed since presentation of the message 185′. The application 120 may compare the elapsed time with a time limit for the variant 225′ of the message 185′. When the elapsed time exceeds the time limit, the application 120 may generate the response 315′ to indicate no user interaction with the variant 225′ of the message 185′. Upon generation, the application 120 may provide, transmit, or otherwise send the response 315′ to the session management service 105.

The response handler 165 may retrieve, identify, or otherwise receive the response 315′ from the user device 110. Upon receipt, the response handler 165 may parse the response 315′ to extract or identify the interaction 305 of the user 210 with the presented variant 225′ of the message 185′. The response handler 165 may record or log the data from the response 315′ into the database 175. The response handler 165 may store an association between the response 315′ and the user profile 180. In some embodiments, the response handler 165 may store and maintain identifications of the variants 225′ of the session in the database 175. The response handler 165 may store an association between the response 315′ and the variant 225′ for the user 210.

In some embodiments, the performance calculator 170 can calculate, generate, or otherwise determine a performance measure 310′ for the variant 225′ of the message 185′. The performance measure 310′ can be a value (e.g., numeric value) identifying or corresponding to the interaction 305 of the user 210 with the presented variant 225′ of the message 185′ on the user interface 145. The performance measure 310′ may be assigned or set to one value when the presented variant 225′ elicits the interaction 305 and another value when the variant 225′ does not elicit an interaction 305. In general, more interactions with the variant 225′ of the message 185′ may result in a higher performance measure 310′, whereas less or no interactions with the variant 225′ of the message 185′ when presented may result in a lower performance measure 310′. In some embodiments, the performance measure 310′ may correspond to a rate of correct or proper responses by the user 210 to the variant 225′ of the message 185′. The performance calculator 170 can analyze information pertaining to the responses 315′ and the variants 225′ and can perform statistical calculations. For example, the performance calculator can calculate variances of frequency of responses 315′, relationships between a specific variant 225′ and a specific response 315′ rate, or between a user profile 180 and a variant 225′.

Using the performance measure 310′, the performance calculator 170 may modify or update the selection model 205. As discussed above, the selection model 205 may keep track of the selection probability for each variant 225′, the performance measure for each variant 225′, and the statistical variance of the performance for each variant 225′, among others. The performance calculator 170 may update the selection model 205 to change the selection probability 220′ of the variant 225′A based on the performance measure 310′. In addition, the performance calculator 170 may modify or update the user profile 180 to identify or include the performance measure 310′, selection probabilities 220, or other statistics associated with the user 210 represented by user profile 180. The performance calculator 170 may maintain information pertaining to the user 210 on the user profile 180 in the database 175. The user profile 180 may identify the behavioral endpoint of the user, a log record of the responses 315 associated with the user, or the set of performance measures 310′ associated with the user 210 for the variants 225′ presented to the user 210. The performance calculator 170 may update the selection model 205 based on information within the user profile 180, the performance measure 310′, or a combination thereof. In some embodiments, the performance calculator 170 may forego calculation of a performance measure 310′ during the second time period for the session.

In some embodiments, the selection model 205 developed may no longer fit with a preference of the user 210. For example, a user 210 may provide a response 315′ that is outside of the selection model's estimations or which exceeds a threshold variance within the selection model 205. Therefore, it may be necessary to modify or change the selection model 205. The selection model 205 may need to be modified responsive to a change in the variants 225 chosen by the user 210 resulting in a statistical deviation or variance exceeding a threshold amount. The performance calculator 170 may determine that a performance measure 310 of a variant during the second time period T_{B} exceeds a threshold. For example, a variance of the response 315 to the message 185 may exceed a threshold. The selection model 205 may need to be edited as a result of changes to user inputs. For example, a user may add or remove data pertinent to the user's demographic information which can affect the selection probability 220 of a variant 225. For example, a user may not have had a pre-diabetes condition listed during time period T_{A}, but subsequently lists a pre-diabetes condition through the user interface 145 in time period T_{B}. Subsequently, the performance calculator 170 may recalculate performance measures 310 associated with one or more variants 225.

In some embodiments, the performance calculator 170 may provide a dashboard interface for presentation. The dashboard interface may be presented on a display of the session management service 105 or a computing device communicatively coupled with the session management service 105. The dashboard interface can provide a presentation of one or more statistics for the session based on the responses received from the user device 110 associated with the user 210. For example, the dashboard interface can display or present the individual responses 315 from the user 210, the performance measure 310 associated with each variant 225 of the message 185, the selection probability and statistical variance indicated by the selection model 205, and information associated with the user 210, among others. The dashboard interface can exhibit the change in the selection model 205 during different time periods (e.g., T_{A} or T_{B}). The dashboard interface can appear as a graphical user interface (e.g., GUI), a table, a series of graphs, or another display medium on the server. The dashboard interface can change dynamically as responses 315 are gathered by the response handler 165.

In this manner, the session management service 105 may provide control of transmission of messages in the networked environment. By updating the selection model 205 using response data during the first time period, the session management service 105 may be able to identify the variant 225 of the message 185 with the highest performance measure for the particular user 210. In doing so, the session management service 105 may reduce computational resources across the system 100 from providing variants 225 of messages 185 most likely to prompt engagement with or by a user 210. In addition, the session management service 105 may overall reduce wasted transmission of instructions 215 for messages 185 that would have otherwise elicited no interaction by the user 210. From selecting variants 225 of messages 185 in this manner, the session management service 105 may also improve the quality of human-computer interaction (HCI) between the user 210 and the user interface 145 of the application 120 on the user device 110.

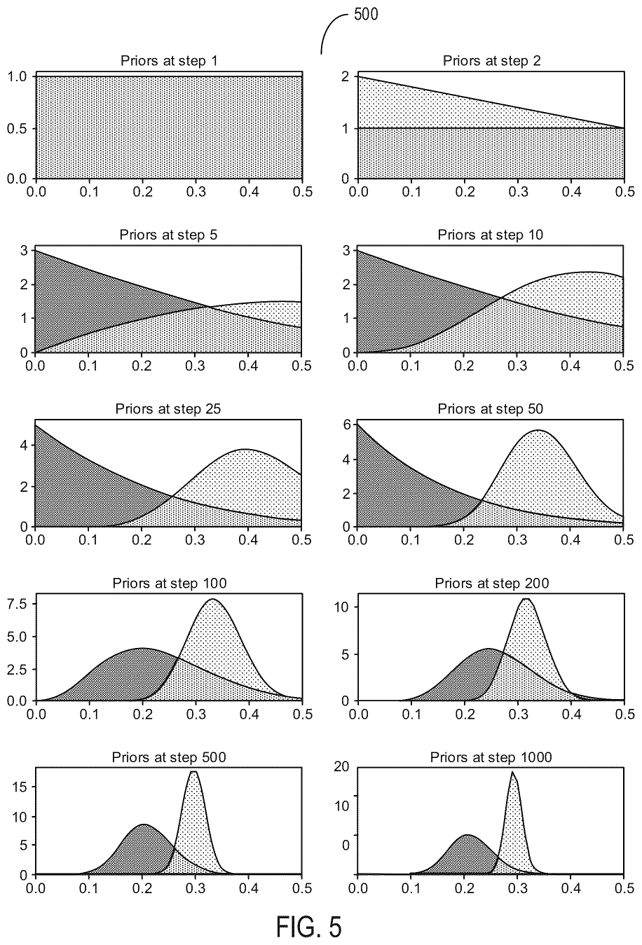

Referring now to FIG. 5, depicted is a set of example graphs 500 for prior probabilities of message variants. These examples as depicted can relate to the iterative development of a selection model 205, wherein each variant 225 of the set of variants has an equal selection probability 220. As responses 315 are received from the user 210, the prior probabilities (e.g., selection probability 220) of each variant can change and converge on a final distribution as time period T_{A} concludes.

In an illustrative example, a variant 225A and 225B can be represented by the plots depicted in FIG. 5. Each variant can be sent to user 210 over time period T_{A}. In this example, variant 225A may be more engaging to user 210 than variant 225B and may have an actual interaction rate of 30% whereas variant 225B may have an interaction rate around 20%. Initially, there may be no reason to believe that either one of the variants is more engaging, so each user of a set of users can receive either variant with equal probability.

As the response handler 165 gathers more response 315, it may emerge that variant 225A seems to have a slightly higher interaction rate. Therefore, the session management service 105 can associate variant 225A with a higher probability (i.e. more frequently than variant 225B). However, the session manager 155 may not stop transmitting variant 225B until the system is statistically confident that variant 225A is preferred. In this manner, more engaging messages can be favored.

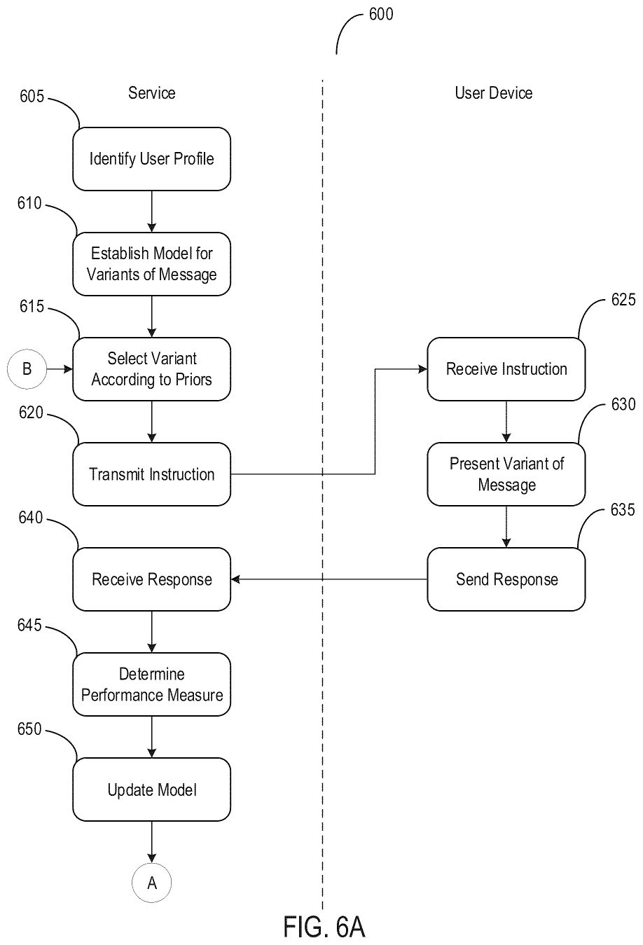

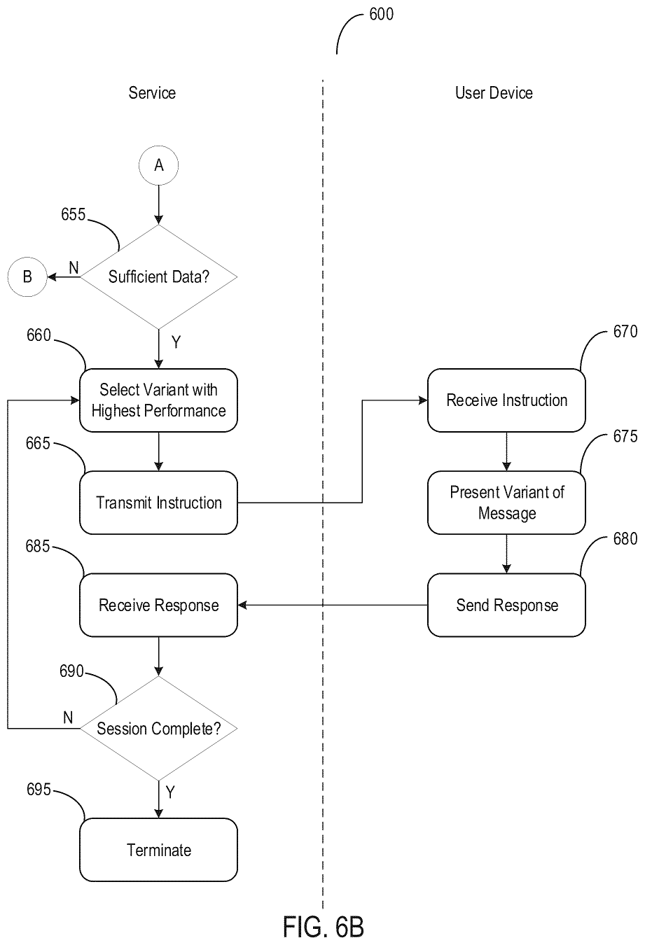

Referring now to FIGS. 6A and 6B, depicted is method 600 for predicting variants of a message to transmit to a client based on the client's responses. Each figure depicts operations performed by the client and operations performed by the service. The method 600 may be implemented or performed using any of the components detailed herein, such as the session management service 105 and the user device 110. Starting from FIG. 6A, under method 600, a service (e.g., the session management service 105) may identify a user profile (e.g., the user profile 180) (605). The service may establish a model (e.g., the selection model 205) for variants (e.g., the set of variants 225) of a message (e.g., the message 185) (610). The service may select a variant (e.g., the variant 225) according to priors (e.g., selection probability 220) (615). The service may transmit an instruction (e.g., the instruction 215) for the variant of the message to a user device (e.g., the user device 110) (620). The user device may receive the instruction (e.g., the instruction 215) (625). The user device may present the variant of the message identified in the instruction (630). The user device may send a response (e.g., the response 315) to the service (635). The service may receive the response from the user device (640). The service may determine performance measures (e.g., the performance measure 310) (645). The service may update the model (650).

Moving onto FIG. 6B, the service may check whether there is sufficient data (655). The determination may be based on an amount of time elapsed in a session or a degree of variance in the model for the variants. If there is not sufficient data, the service may proceed back to (615) and repeat the method 600. If there is sufficient data, the service may select the variant with the highest performance (660). The service may transmit an instruction (e.g., the instruction 215′) including the selected variant of the message (665). The client may receive the instruction from the service (670). The user device may present the variant of the message (675). The user device may send a response (e.g., the response 315′) to the server (680). The service may receive the response from the user device (685). The service may check whether the session is complete (690). If the session is not complete, the service may proceed back to (660) and repeat the method 600. If the session is complete, the service may terminate the session (695).

### B. Network and Computing Environment

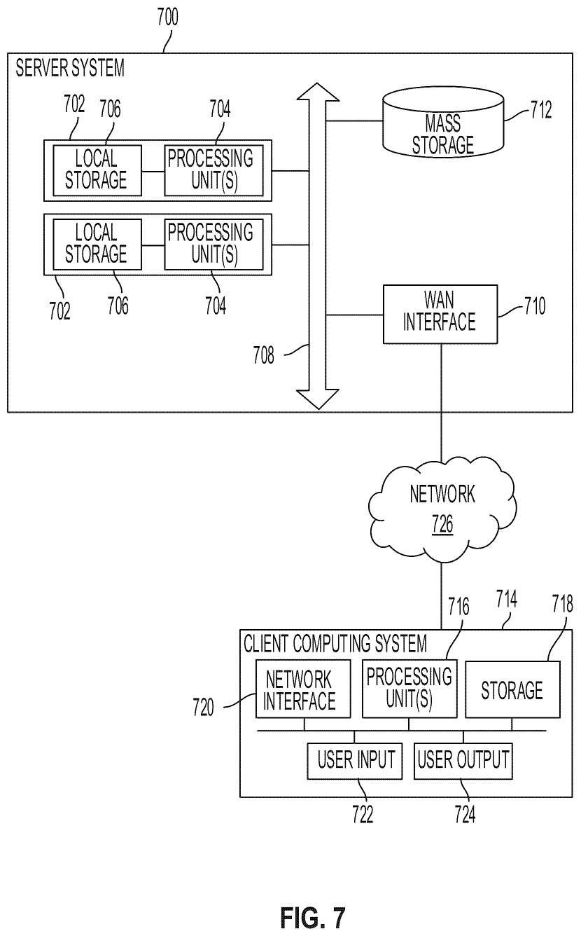

Various operations described herein can be implemented on computer systems. FIG. 7 shows a simplified block diagram of a representative server system 700, client computer system 714, and network 726 usable to implement certain embodiments of the present disclosure. In various embodiments, server system 700 or similar systems can implement services or servers described herein or portions thereof. Client computer system 714 or similar systems can implement clients described herein. The system 100 described herein can be similar to the server system 700. Server system 700 can have a modular design that incorporates a number of modules 702 (e.g., blades in a blade server embodiment); while two modules 702 are shown, any number can be provided. Each module 702 can include processing unit(s) 704 and local storage 706.

Processing unit(s) 704 can include a single processor, which can have one or more cores, or multiple processors. In some embodiments, processing unit(s) 704 can include a general-purpose primary processor as well as one or more special-purpose co-processors such as graphics processors, digital signal processors, or the like. In some embodiments, some or all processing units 704 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 704 can execute instructions stored in local storage 706. Any type of processors in any combination can be included in processing unit(s) 704.

Local storage 706 can include volatile storage media (e.g., DRAM, SRAM, SDRAM, or the like) and/or non-volatile storage media (e.g., magnetic or optical disk, flash memory, or the like). Storage media incorporated in local storage 706 can be fixed, removable, or upgradeable as desired. Local storage 706 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 704 need at runtime. The ROM can store static data and instructions that are needed by processing unit(s) 704. The permanent storage device can be a non-volatile read-and-write memory device that can store instructions and data even when module 702 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 706 can store one or more software programs to be executed by processing unit(s) 704, such as an operating system and/or programs implementing various server functions such as functions of the system 100 or any other system described herein, or any other server(s) associated with system 100 or any other system described herein.

“Software” refers generally to sequences of instructions that, when executed by processing unit(s) 704, cause server system 700 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 704. Software can be implemented as a single program or a collection of separate programs or program modules that interact as desired. From local storage 706 (or non-local storage described below), processing unit(s) 704 can retrieve program instructions to execute and data to process in order to execute various operations described above.

In some server systems 700, multiple modules 702 can be interconnected via a bus or other interconnect 708, forming a local area network that supports communication between modules 702 and other components of server system 700. Interconnect 708 can be implemented using various technologies, including server racks, hubs, routers, etc.

A wide area network (WAN) interface 710 can provide data communication capability between the local area network (e.g., through the interconnect 708) and the network 726, such as the Internet. Other technologies can be used to communicatively couple the server system with the network 726, including wired (e.g., Ethernet, IEEE 802.3 standards) and/or wireless technologies (e.g., Wi-Fi, IEEE 802.11 standards).

In some embodiments, local storage 706 is intended to provide working memory for processing unit(s) 704, providing fast access to programs and/or data to be processed while reducing traffic on interconnect 708. Storage for larger quantities of data can be provided on the local area network by one or more mass storage subsystems 712 that can be connected to interconnect 708. Mass storage subsystem 712 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 712. In some embodiments, additional data storage resources may be accessible via WAN interface 710 (potentially with increased latency).

Server system 700 can operate in response to requests received via WAN interface 710. For example, one of modules 702 can implement a supervisory function and assign discrete tasks to other modules 702 in response to received requests. Work allocation techniques can be used. As requests are processed, results can be returned to the requester via WAN interface 710. Such operation can generally be automated. Further, in some embodiments, WAN interface 710 can connect multiple server systems 700 to each other, providing scalable systems capable of managing high volumes of activity. Other techniques for managing server systems and server farms (collections of server systems that cooperate) can be used, including dynamic resource allocation and reallocation.

Server system 700 can interact with various user-owned or user-operated devices via a wide-area network such as the Internet. An example of a user-operated device is shown in FIG. 7 as client computing system 714. Client computing system 714 can be implemented, for example, as a consumer device such as a smartphone, other mobile phone, tablet computer, wearable computing device (e.g., smart watch, eyeglasses), desktop computer, laptop computer, and so on.

For example, client computing system 714 can communicate via WAN interface 710. Client computing system 714 can include computer components such as processing unit(s) 716, storage device 718, network interface 720, user input device 722, and user output device 724. Client computing system 714 can be a computing device implemented in a variety of form factors, such as a desktop computer, laptop computer, tablet computer, smartphone, other mobile computing device, wearable computing device, or the like.

Processing unit 716 and storage device 718 can be similar to processing unit(s) 704 and local storage 706 described above. Suitable devices can be selected based on the demands to be placed on client computing system 714; for example, client computing system 714 can be implemented as a “thin” client with limited processing capability or as a high-powered computing device. Client computing system 714 can be provisioned with program code executable by processing unit(s) 716 to enable various interactions with server system 700.

Network interface 720 can provide a connection to the network 726, such as a wide area network (e.g., the Internet) to which WAN interface 710 of server system 700 is also connected. In various embodiments, network interface 720 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 722 can include any device (or devices) via which a user can provide signals to client computing system 714; client computing system 714 can interpret the signals as indicative of particular user requests or information. In various embodiments, user input device 722 can include any or all of a keyboard, touch pad, touch screen, mouse or other pointing device, scroll wheel, click wheel, dial, button, switch, keypad, microphone, and so on.

User output device 724 can include any device via which client computing system 714 can provide information to a user. For example, user output device 724 can include display-to-display images generated by or delivered to client computing system 714. The display can incorporate various image generation technologies, e.g., a liquid crystal display (LCD), light-emitting diode (LED) display including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 724 can be provided in addition to or instead of a display. Examples include indicator lights, speakers, tactile “display” devices, printers, and so on.

Some embodiments include electronic components, such as microprocessors, storage, and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 704 and 716 can provide various functionality for server system 700 and client computing system 714, including any of the functionality described herein as being performed by a server or client, or other functionality.

It will be appreciated that server system 700 and client computing system 714 are illustrative and that variations and modifications are possible. Computer systems used in connection with embodiments of the present disclosure can have other capabilities not specifically described here. Further, while server system 700 and client computing system 714 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies, including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of controlling transmissions of messages to users, comprising:
transmitting, by a server, over a first time period of a session for a user towards achieving a behavioral endpoint, a plurality of first instructions to a user device associated with the user, each of the plurality of first instructions to present a message in at least one of a plurality of variants;
receiving, by the server, from the user device over the first period, a plurality of responses corresponding to the plurality of first instructions, each of the plurality of responses by the user to a respective presentation of the message in at least one of the plurality of variants towards achieving the behavioral endpoint;
determining, by the server, responsive to elapsing of the first time period of the session, a respective performance measure of the message in each variant of the plurality of variants using the plurality of responses;
selecting, by the server, for a second time period of the session, a variant from the plurality variants of the message based on the performance measure for the variant; and
transmitting, by the server, during the second time period, a second instruction to the user device associated with the user to present the message in the selected variant.

2. The method of claim 1, further comprising:
determining, by the server, that a second performance measure of the message in the selected variant during the second time period is different from the respective performance metric during the first time period by a threshold margin; and
selecting, by the server, responsive to determining that the second performance measure is different, a second variant from the plurality of variants for the message to transmit.

3. The method of claim 1, further comprising selecting, by the server, for a third time period of the session, a second variant of a plurality of second variants for a second message to transmit based on the selection of the variant for the message for the second time period.

4. The method of claim 1, further comprising establishing, by the server, using the plurality of responses, a model to track, for each variant of the plurality of variants, (i) the respective performance measure identifying a degree of interactivity by the user with the message in the corresponding variant and (ii) a variance of the performance measure.

5. The method of claim 1, further comprising determining, by the server, to select one of the plurality of variants of the message for the second time period, responsive to determining that a variance of the performance measure is below a threshold.

6. The method of claim 1, further comprising determining, by the server, based on a profile of the user, a plurality of prior probabilities of selecting the corresponding plurality of variants of the message for transmission during the first time period of the session.

7. The method of claim 1, further comprising providing, by the server, a dashboard interface for presentation identifying one or more statistics for the session based on the plurality of responses from the user device associated with the user.

8. The method of claim 1, wherein determining the respective performance measure further comprises determining the respective performance measure of the message in each variant of the plurality of variants using the plurality of responses and a profile of the user identifying information for the session.

9. The method of claim 1, wherein each variant of the plurality of variants for the message comprises at least one of: (i) an identification of a type of an action to be performed by the user via the user device, (ii) an identification of a theme for content to be presented via the user device, (iii) an identification of a type of content to be presented via the user device, (iv) an identification of inclusion of user information, (v) a definition of a respective layout for the content, (vi) an identification corresponding to a personality associated with the user, (v) an identification of a tone of the content to be presented via the user device, (vi) an identification of content based on the literacy of the user, (vii) an identification of content based on the health knowledge of the user, (viii) an identification of a learning style associated with the user, (ix) an identification of a personal incentive or motivation of the user, (x) an identification based on the messaging style of the user, (xi) an identification of values of the user, or (xii) a cognitive ability of the user.

10. The method of claim 1, further comprising maintaining, by the server, on a database, a profile of the user identifying (i) the behavioral endpoint to be achieved, (ii) a log record for the plurality of responses from the user, and (iii) a plurality of performance metrics for the plurality of variants of the message.

11. The method of claim 1, wherein the user is on a medication associated with addressing the behavioral endpoint, at least in partial concurrence with the session.

12. A system for controlling transmissions of messages to users, comprising:
at least one server having one or more processors coupled with memory, configured to
transmit, over a first time period of a session for a user towards achieving a behavioral endpoint, a plurality of first instructions to a user device associated with the user, each of the plurality of first instructions to present a message in at least one of a plurality of variants;
receive, from the user device over the first period a plurality of responses corresponding to the plurality of first instructions, each of the plurality of responses by the user to a respective presentation of the message in at least one of the plurality of variants towards achieving the behavioral endpoint;
determine, responsive to elapsing of the first time period of the session, a respective performance measure of the message in each variant of the plurality of variants using the plurality of responses;
select, for a second time period of the session, a variant from the plurality variants of the message based on the performance measure for the variant; and
transmit, during the second time period, a second instruction to the user device associated with the user to present the message in the selected variant.

13. The system of claim 12, wherein the at least one server is further configured to:
determine that a second performance measure of the message in the selected variant during the second time period is different from the respective performance metric during the first time period by a threshold margin; and
select, responsive to determining that the second performance measure is different, a second variant from the plurality of variants for the message to transmit.

14. The system of claim 12, wherein the at least one server is further configured to select, for a third time period of the session, a second variant of a plurality of second variants for a second message to transmit based on the selection of the variant for the message for the second time period.

15. The system of claim 12, wherein the at least one server is further configured to establish, using the plurality of responses, a model to track, for each variant of the plurality of variants, (i) the respective performance measure identifying a degree of interactivity by the user with the message in the corresponding variant and (ii) a variance for the performance measure.

16. The system of claim 12, wherein the at least one server is further configured to determine to select one of the plurality of variants of the message for the second time period, responsive to determining that a variance of the performance measure is below a threshold.

17. The system of claim 12, wherein the at least one server is further configured to determine, based on a profile of the user, a plurality of prior probabilities of selecting the corresponding plurality of variants of the message for transmission during the first time period of the session.

18. The system of claim 12, wherein the at least one server is further configured to provide a dashboard interface for presentation identifying one or more statistics for the session based on the plurality of responses from the user device associated with the user.

19. The system of claim 12, wherein the at least one server is further configured to determine the respective performance measure of the message in each variant of the plurality of variants using the plurality of responses and a profile of the user identifying information for the session.

20. The system of claim 12, wherein each variant of the plurality of variants for the message comprises at least one of: (i) an identification of a type of an action to be performed by the user via the user device, (ii) an identification of a theme for content to be presented via the user device, (iii) an identification of a type of content to be presented via the user device, (iv) an identification of inclusion of user information, (v) a definition of a respective layout for the content, (vi) an identification corresponding to a personality associated with the user, (v) an identification of a tone of the content to be presented via the user device, (vi) an identification of content based on the literacy of the user, (vii) an identification of content based on the health knowledge of the user, (viii) an identification based on a learning style associated with the user, (ix) an identification of a personal incentive or motivation of the user, (x) an identification of a messaging style of the user, (xi) an identification of values of the user, or (xii) a cognitive ability of the user.

21. The system of claim 12, wherein the at least one server is further configured to maintain, on a database, a profile of the user identifying (i) the behavioral endpoint to be achieved, (ii) a log record for the plurality of responses from the user, and (iii) a plurality of performance metrics for the plurality of variants of the message.

22. The system of claim 12, wherein the user is on a medication associated with addressing the behavioral endpoint, at least in partial concurrence with the session.
